(19) United States
(12) Patent Application Publication
(10) Pub. No.: US 2004/0247689 A1
(43) Pub. Date: Dec. 9, 2004

(54) Process for preparing a pharmaceutical active ingredient with high specific surface area

(76) Inventor: Guy Samburski, Ganot-Hadar (IL)

Correspondence Address: KENYON & KENYON, One Broadway, New York, NY, 10004-1050, US

(21) Appl. No.: 10/809,754

(22) Filed: Mar. 25, 2004

Related U.S. Application Data
(60) Provisional application No. 60/458,083, filed on Mar. 26, 2003.

(51) Int. Cl. A61K 9/58; A61K 9/14

(52) U.S. Cl. 424/489

(57) ABSTRACT

Provided is a process for micronization of active pharmaceutical ingredients to provide active pharmaceutical ingredients with a high specific surface area.

### PRIORITY

This application claims the benefit of U.S. provisional application Serial No. 60/458,083, filed Mar. 26, 2003, the content of which is incorporated herein.

### FIELD OF THE INVENTION

The present invention relates to the specific surface area of active pharmaceutical ingredients.

### BACKGROUND OF THE INVENTION

Formulation of an active pharmaceutical ingredient may be problematic, particularly when the active pharmaceutical ingredient is designed for inhalation, has low bioavailability or is used in extended release formulations. Three active pharmaceutical ingredients, nifedipine, salmeterol and leuprolide are used in the present application to illustrate such problems.

Nifedipine is a calcium ion influx inhibitor (slow-channel blocker or calcium ion antagonist) which inhibits the transmembrane influx of calcium ions into vascular smooth muscle and cardiac muscle. Nifedipine is prescribed for the treatment of hypertension. Nifedipine, also known as 1,4-dihydro-2,6-dimethyl-4-(2-nitrophenyl)-dimethyl ester, has the following structure:

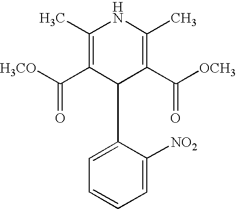

and is marketed in the U.S under the tradmemarks Adalat® and Procardia®, among others. Nifedipine is disclosed and claimed in numerous patents. For example, U.S. Pat. No. 4,412,986 is directed to a solid formulation of nifedipine having high bioavailability and reduced bulk composed of 1 part nifedipine and 1-20 parts polyvinylpyrrolidone, methyl cellulose, hydroxypropyl cellulose and hydroxypropylmethyl cellulose; U.S. Pat. No. 4,665,081 is directed to a dry particulate nifedipine composition made by co-pulverization of nifedipine, casein and certain specified inorganic excipients in absence of a liquid vehicle; U.S. Pat. No. 4,880,623 is directed to a Solid finely divided nifedipine composition obtained by mixing micronized nifedipine, micronized inert excipient and a polyethylene glycol; U.S. Pat. No. 4,882,144 is directed to a solid, rapidly absorbable nifedipine composition containing PVP, cellulose, starch, and cross-linked, insoluble PVPP; U.S. Pat. No. 4,904,699 is directed to a liquid nifedipine concentrate stabilized against light; U.S. Pat. No. 4,933,186 is directed to a tablet composed of (a) a rapid release core of nifedipine, (b) a coating having no nifedipine, and (c) a rapid release nifedipine coating; U.S. Pat. No. 4,954,346 is directed to a gelatin capsule filled with a liquid nifedipine composition containing a carbonate and a surfactant; U.S. Pat. No. 4,966,772 is directed to a delayed release nifedipine composition comprising (a) A core containing at least 50% of nifedipine in delayed release form, (b) A slow dissolving coating containing no nifedipine made of a hydrophilic gel, and (c) A rapid release coating of nifedipine; U.S. Pat. No. 5,229,116 for co-administration of nifedipine and a flavinoid (such as that in grape juice) to prolong bio-availability by inhibiting cytochrome P 450 oxidation; U.S. Pat. No. 5,543,099 for granulation of inactive ingredients, such as hydroxypropyl cellulose, with active ingredients, such as nifedipine; followed by micronization of the granules. U.S. Publ. No. 2003/0091626 is directed to an orally disintegrating preparation of nifedipine. All these patents and applications are incorporated herein by reference.

Various U.S. patents are also directed to methods of use of nifedipine. U.S. Pat. No. 4,582,840 is directed to use of nifedipine for combating sodium balance renal insufficiency; U.S. Pat. No. 4,690,935 for inhibition of tumor growth, U.S. Pat. No. 4,728,660 for treatment of thromboelmbolic disease, U.S. Pat. No. 4,851,404 for treatment of sickle cell anemia, U.S. Pat. No. 4,918,076 for treatment of alcohol addiction, U.S. Pat. No. 4,978,533 for treatment of coronary insufficiency, U.S. Pat. No. 5,053,419 for treatment of AIDS dimentia complex, U.S. Pat. No. 5,071,642 for use of claimed formulation to treat coronary heart disease or high blood pressure, U.S. Pat. No. 5,124,340 for treatment for cocaine addiction, U.S. Pat. No. 5,145,859 for treatment of interstitial cystitis and urethral syndrome. All these patents are incorporated herein by reference.

Nifedipine is a yellow crystalline substance, soluble in acetone, but practically insoluble in water. The lack of solubility of nifedipine creates a problem since bioavailability of a water insoluble active ingredient is usually poor. An approach to increasing bioavailability of an active pharmaceutical ingredient is through manipultation of specific surface area. Nifedipine, as well as other active pharmaceutical ingredients, may possess a certain Specific Surface Area (S.S.A.), which may affect their bioavailability. (See generally Lantz, Russel J.Jr, size reduction, in Lachman Leon & Lieberman Herbert A. Pharmaceutical dose forms Vol. 2, p. 77-152, incorporated herein by reference) (Hereinafter “Lantz”).

Nifedipine with specific surface area is disclosed and claimed in U.S. Pat. No. 5,264,446. U.S. Pat. No. 5,264,446 is directed to a sustained release formulation comprising nifedipine crystals having a specific surface area of 1.0-4 m²/g. The '446 patent is incorporated herein by reference. Nifedipine with a high surface area has also been subject of litigation in the United States. See Bayer A G v. Biovail Co., 279 F.3d 1340 (Fed. Cir. 2002).

Another active pharmaceutical ingredient is salmeterol, disclosed in U.S. Pat. No. 4,992,474, and marketed under the name SERVENT and ADVAIR as a xinofoate salt. According to the maker of SERVENT, salmeterol xinafoate is a white to off-white powder that is freely soluble in methanol; slightly soluble in ethanol, chloroform, and isopropanol; and sparingly soluble in water. Salmetero xinafoate has the following structure:

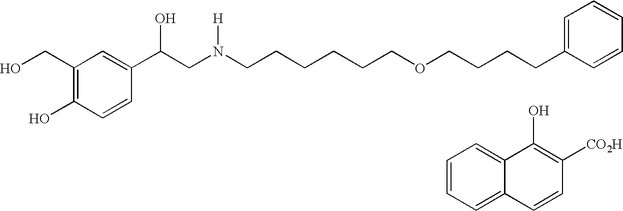

Salmetero xinofoate is a bronchodilator for treatment of obstructive lung conditions such as asthma, emphysema and chronic brochitis. It is administrated as a powder for inhalation. The inhalable particle should be in size of less than 3 icrons for absorption. Micronization of salmeterol xinofoate by traditional methods and their adverse effects are discussed in Shekunov. B. Y. et al “Physical properties of supercritically-processed and micronised powders for respiratory drug delivery” in KONA No. 20, 2002, incorporated herein by reference.

Another pharmaceutical active ingredient is leuprolide, which is marketed under the name LUPRON as an acetate salt. According to the maker of LUPRON, leupromide is an injectable synthetic nonapeptide analog of naturally occurring gonadotropin releasing hormone (GnRH or LH-RH) and has the chemical name 5-Oxo-L-prolyl-L-histidyl-L-tryptophyl-L-seryl-L-tyrosyl-D-leucyl-L-leucyl-L-arginyl-N-ethyl-L-prolinamide acetate (salt). Micronization, by increasing solubility, makes injectables APIs more suitable. Also peptides may be used for systemic inhalation therapy. In order to obtain good absorption of the the active pharmaceutical ingredient in the lungs, the active pharmaceutical ingredient should be micronized to size smaller than 3 icrons. Conventional micronization may not be efficient and might damage the active pharmaceutical ingredient.

Specific Surface area is defined in units of square meters per gram (m²/g). It is usually measured by nitrogen absorption analysis (See Lantz). In this analysis, nitrogen is absorbed on the surface of the substance. The amount of the absorbed nitrogen (as measured during the absorption or the subsequent desorption process) is related to the surface area via a formula known as the B.E.T. formula. An instrument by Strohlein (for example model areameter 2) is mentioned during prosecution of U.S. Pat. No. 5,264,446. Other commercial instrument are manufactured by quantachrome (for example model monosorb) or Coulter (for example model SA3100. ) The analysis may be performed in a single test (single point measurement) or in a series of tests in various nitrogen pressures (multipoint measurement).

S.S.A. of an active pharmaceutical ingredient may be affected by various factors. There is a general connection between Specific Surface Area and Particle Size Distribution (P.S.D.); the smaller the Particle Size Distribution, the higher the Specific Surface Area. Additional factors affecting S.S.A. are the particle shape, the particle porosity, and inter-particle binding forces known to create aggregation or agglomeration. The following table, based on experiments performed by the applicants, describes the relation between the S.S.A. and the mean P.S.D., d(0.5) of nifedipine:

[image]

It has been shown that nifedipine with a high surface area has greater bioavailability. See e.g. U.S. Pat. No. 5,264,446. According to the seventh edition of Pharmaceutical Dosage Forms and Drug Delivery Systems, other active pharmaceutical ingredients that show greater bioavailability upon micronization include theophylline, griseofluvin, sulfisoxazole and nitrofurantoin. There is a need in the art to prepare active pharmaceutical ingredients such as nifedipine with a high surface area to obtain formulations with greater bioavalability, and to compensate for any loss of surface area before formulation. Micronizaton also helps in absorption of inhaled active pharmaceutical ingredients.

Micronization also allows for formulation of such active ingredients for extended release. For example, when an active pharmaceutical ingredient is used in extended release formulations, it is possible with the process of the present invention to put the API on a plurality of pellets, with some pellets containing micronized API, while others non-micornized API. See e.g. Ansel et al, Pharmaceutical Dosage Forms And Drug Delivery Systems, 7^{th} ed. Page 232. It is also possible to use a hydrogel, such as with HPMC, containing micronized and non-micronized particles, where the micronized particles escape the hydrogel at a faster rate. In some situations, only micronized particles may be used with a hydrogel, where non-micronized particles would be too large to escape the hydrogel effectively over time. There is a need in the art for effective micronization of pharmaceutical active ingredients.

### SUMMARY OF THE INVENTION

In one aspect, the present invention provides a process for preparing an active pharmaceutical ingredient having a specific surface area of at least about 5.0 m²/g as measured by B.E.T. comprising storing the active pharmaceutical ingredient at a temperature of below about 0° C. and micronizing the active pharmaceutical ingredient to obtain a specific surface area of at least about 5.0 m²/g.

In another aspect, the present invention provides a process for preparing nifedipine having a specific surface area of at least about 5.0 m²/g as measured by B.E.T. comprising storing nifedipine powder for a first time at a temperature below about 0° C. for at least about 4 hours, micronizing the nifedipine for a first time to obtain a specific surface area of about 5.0 m²/g to about 6.0 m²/g, as measured by B.E.T., storing the nifedipine for a second time at a temperature below about −10° C. and micronizing the nifedipine of step c for a second time to obtain an specific surface area of about 6.0 m²/g to about 7.0 m²/g, as measured by B.E.T.

In another aspect, the present invention provides a process for maintaining specific surface area of an active pharmaceutical ingredient having a specific surface area of at least about 5.0 m²/g as measured by B.E.T. comprising the step of storing the active pharmaceutical ingredient at a temperature of below about −10° C., wherein the active pharmaceuticcal ingredient retains a specific surface area within about 0.5 m²/g after at least about six months, as measured by B.E.T.

In another aspect, the present invention provides a process for preparing a pharmaceutical oral dosage form comprising storing an active pharmaceutical ingredient having a particle size distribution of about 15 to about 30 icrons for a first time at a temperature of below about 0° C. for at least about 4 hours, micronizing the stored active pharmaceutical ingredient for a first time to obtain an S.S.A. of at least about 5.5 m²/g, as measured by B.E.T., storing the active pharmaceutical ingredient for a second time at a temperature of below about −10° C., micronizing the active pharmaceutical ingredient for a second time to obtain a specific surface area of at least about 6.5 m²/g, as measured by B.E.T., storing the active pharmaceutical ingredient at a temperature of below about −10° C. for a second time and converting the active pharmaceutical ingredient to a pharmaceutical oral dosage form.

In another aspect, the present invention provides a process for preparing an active pharmaceutical ingredient selected from the group consisting of nifedipine, salmeterol and leuprolide, having a specific surface area of at least about 5.5 m²/g as measured by B.E.T. comprising storing the active pharmaceutical ingredient at a temperature of below about 0° C. for at least about 4 hours and micronizing the stored active pharmaceutical ingredient.

In another aspct, the present invention provides a process for preparing an active pharmaceutical ingredient comprising storing the active pharmaceutical ingredient at a temperature of below about 0° C. for at least about 24 hours and micronizing the active pharmaceutical ingredient, wherein the storing results in a minimum increase of about 0.5 m/g2 in specific surface area compared to micronizing without storing (such as storage at room temperature).

In another aspect, the present invention provides a process for preparing a pharmaceutical oral dosage form comprising storing an active pharmaceutical ingredient for a first time at a temperature of below about negative 10° C. for at least about 24 hours, micronizing the stored active pharmaceutical ingredient at a feed rate of about 20 kg/hr and a feed air pressure of about 8 bar to about 8.5 bar for a first time to obtain an S.S.A. of at least about 5.5 m²/g, as measured by B.E.T., storing the active pharmaceutical ingredient for a second time at a temperature of below about −10° C., micronizing the stored active pharmaceutical ingredient for a second time at a feed rate of about 20 kg/hr and a feed air pressure of about 8 bar to about 8.5 bar to obtain an S.S.A. of at least about 6.5 m²/g, as measured by B.E.T., storing the active pharmaceutical ingredient at a temperature of below about −10° C. for a second time and converting the active pharmaceutical ingredient to a pharmaceutical oral dosage form.

### DETAILED DESCRIPTION OF THE INVENTION

As used herein, the term “micronization” refers to a decrease in particle size through application of force to a particle, resulting in the break-up of the particle. Such force may be applied by collision of particles at high speeds.

The present invention provides a process for preparing an active pharmaceutical ingredient, particularly those that are used for inhalation, have low bioavailability and are used in extended release formulations, with a high surface area, i.e., at least about 4.0 m²/g. Preferably, the active pharmaceutical ingredient of the present invention has an S.S.A. of more than about 5.0 m²/g, more preferably, more than about 5.5 m²/g, and most preferably, more than about 6.5 m²/g. In one embodiment, the S.S.A. is from about 5.0 m²/g to about 7.0 m²/g, more preferably from about 6.0 m²/g to about 7.0 m²/g.

The term active pharmaceutical ingredient (API) in the present invention encompasses small organic molecules and peptides, their salts and esters, and refers to the ingredient having physiological activity after administration to a mammal. Adjuvants (when a secondary active pharmaceutical ingredient is administered) are included within the term active pharmaceutical ingredient. Preferred API include nifedipine, leuprolide and salmeterol, with nifedipine being most preferred.

In the process of the present invention, an active pharmaceutical ingredient is stored at a temperature of below 0° C., preferably below about −10° C., most preferably of about −10° C. and about −20° C. for a sufficient time to allow for increasing the specific surface area upon micronization. Preferably the storage is carried out for such time that an increase of 0.5 m²/g over micronization without storage is obtained. Storage is preferably carried out until the API reaches the temperture of the freezer, preferably for at least about 4 hours, more preferably for at least about 24 hours. The storage could for example be for about a day, about 2-3 days, about a week, or about a month. The cooled API is then micronized within a reasonable time after being taken out of the freezer, preferably with an immediate intial feed, such as in less than about 30 minutes, more preferably in less than about 15 minutes, and most preferably in less than about 1-5 minutes, so that a substantial loss in temperature of the API does not occur before micronization.

After micronization, the micronized API is preferably kept at a temperature of below about −10° C. to maintain the obtained surface area. More preferably, the API is stored at a temperature of between about −10° C. and about −20° C.

Micronization and storage at such temperature may further protect the API against chemical and/or physical degradation.

The feed rate for micronization is preferably of about 4 kg/hr to about 30 kg/hr, most preferably, the feed rate is about 20 kg/hr. The feed air pressure is preferably of about 2 bar to about 10 bar, most preferably the feed air pressure is of about 8 bar to about 8.5 bar.

Preferably, the API micronized is a powder in the solid state and may be in the form of crystals, powder aggregates and course powder, preferably as a fine powder of crystals with particle size distribution of about 10 to about 40 icrons, more preferably about 15 to about 30 icrons. The starting API may have a surface area as low as from about 0.5 m²/g to about 1 m²/g.

The micronization step preferably decreases the size of the stored API to less than about 3μ, preferably to less than about 1μ, and preferably increases the stored API surface area to at least about 5.5 m²/g.

Preferably, the micronization is performed with a fluid-energy mill. One of skill in the art appreciates that a fluid energy mill, or micronizer, is an especially preferred type of mill for its ability to produce particles of small size in a narrow size distribution. As those skilled in the art are aware, micronizers use the kinetic energy of collision between particles suspended in a rapidly moving fluid stream to cleave the particles. An air jet mill is a preferred fluid energy mill. The suspended particles are injected under pressure into a recirculating particle stream. Smaller particles are carried aloft inside the mill and swept into a vent connected to a powder collector,such as a cyclone or a filter bag house.

The storage at a low temperature (below about 0° C. prferred, more preferably below about negative 10° C., and most preferably of about −10° C. and about −20° C.), stabilzes the micronized nifedipine. Preferably, the storage maintains the specific surface area in a suitable range for at least about six months. The freezing of the micronized product immediately after micronization stabilizes the S.S.A., which decreases by less than about 1.0 m²/g, more preferably less than about 0.5 m²/g in about six months. One of skill in the art would appreciate that this rate of decrease is not applicable after formulation since the filler would decrease contact between various particles of the active pharmaceutical ingredient.

In order to obtain initial surface area higher than about 6.5 m²/g, the micronization process is preferably repeated. Re-micronization may be also used to regenerate a decreased surface area. For example, a substance with initial S.S.A. of about 5.5 m²/g, which decreases with time to about 5.0 m²/g, may re-gain an S.S.A. of about 6.5 m²/g after passing the above process (freezing, micronization, re-freezing). It is possible to re-micronize immdiately without further freezing as long as the active pharmaceutical ingredient is at a sufficiently low temperature.

Further steps such as packaging, transportation and long-term storage are preferably performed in temperatures of below about 8° C., more preferably, at below about −10° C., and most preferably of about −10° C. to about −20° C. If all the steps are performed in a temperature of below about −10° C., the API's S.S.A. or that of another active pharmaceutical ingredient may not decrease by more than about 1 m²/g, more preferably 0.5 m²/g in at least about three months, and most preferably in at least about six months.

The pharmaceutical compositions of the present invention, such as oral and inhaled phamaceutical dosage forms, contain nifedipine, leuprolide, salmeterol, theophylline, griseofluvin, sulfisoxazole and nitrofurantoin with a high surface area, optionally in a mixture with other active ingredients. In addition to the active ingredient(s), the pharmaceutical compositions of the present invention may contain one or more excipients. Excipients are added to the composition for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the active pharmaceutical ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

In one embodiment, the capsules, tablets and lozenges, and other unit dosage forms preferably contain about 10 to about 90 g of nifedipine. The capsules preferably contain from about 10 g to about 20 g of nifedipine, while the tablets, preferably in the form of extended release, contain from about 30 g to about 90 g of nifedipine. A preferred host is a mammal, most preferably a human.

Nifedipine may be administered alone or in combination with other hypertensive agents. Preferably nifedipine is administered in a maintenance dose of from about 30 g to about 60 g per day, once a day on an empty stomach.

The formulations of the present invention administered via inhalation contain powder administered by the help of dry-powder inhalers. In addition to the API, these inhaled compositions include inert propelants, and pharmaceutical diluents, such as alpha lactose monohydrate, to aid the formulations flow properties, metering uniformity and to protect the powder agaist effects of humidity (Ansel et al, pg 172). A particularly favored formulation is a diskus containing about 100/50 mcg, 250/50 mcg or 500/50 mcg of fluticasone and salmeterol xinofoate (base equivalent). Salmeterol xinofoate administered is preferably microfine and contains a sugar such as lactose for formulation.

### EXAMPLES

Instrumentation:

Freezer with controlled temperature at −10° C. to −20° C.

Micronizer: Fluid energy mill such as Microgrinding MC-500 KX, Hosokawa Fluidized bed opposed jet mill AFG®, Sturtavent micronizer jet mill.

### Example 1

This example illustrates a process for obtaining an API with initial S.S.A. of at least about 5.5 m²/g:

A sample of nifedipine with an average particle size ditribution of about 20-30 icrons was put in a freezer at a temperature of about −10° C. to about −20° C. for about 24 hours to become embrittled. The resulting API was then put in a micronizer, and micronized at a controlled feed rate and air pressure. For the microgrinding, the feed rate was 20±1 kg/hr, the feed air pressure was 8-8.5 bar and the grinding air was 3-4.5 bar.

The micronized product was immediately transferred back into the freezer. (A package of 10 Kg was used, once the package was full, the package was sealed and transferred to the freezer. Since the feed rate in this example was about 20 Kg/hr, the API went into the freezer within about 30 minutes of micronization.)

### Example 2

This example illustrates a process for obtaining API with initial S.S.A. of at least about 6.5 m²/g:

The process was the same as that illustrated in Example 1, but the micronization process was repeated twice under the same micronization parameters, with the subsequent freezing, to obtain nifedipine with an S.S.A. of at least about 6.5 m²/g.

### Example 3

PROPHETIC-This example illustrates a process for re-generating S.S.A. of at least 5.5 m²/g:

A nifedipine sample whose S.S.A. has decreased is used as a starting API in the process of either examples 1 or 2 to regain or exceed its original S.S.A.

### Example 4

### Comparative Example

Nifedipine with the same characteristics as example 1 was micronized under the same conditions without being put in a freezer. The result was a nifedipine with an S.S.A. of 4.6 m²/g.

### Example 5

PROPHETIC

An active pharmaceutical ingrdient is kept in temperature −10 to −20° C. It is micronized with a micronizer such as microgrinding MC-100 with feed rate of 0.2 kg/hr, feed air pressure of 8 bar and grinding air pressure of 6 bar. The API is stored at −10 to −20° C. after micronization to preserve it from losing its surface properties by mechanisms such as amorphization.

### Example 6

PROPHETIC

An active pharmaceutical ingredient is kept at a temperature of −10 to −20° C. It is micronized with a micronizer such as microgrinding MC-100 with feed rate of 0.2 kg/hr, feed air pressure of 8 bar and grinding air pressure of 6 bar. The API is stored at −10 to −20° C. after micronization to preserve its surface properties.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

What is claimed is:

1. A process for preparing an active pharmaceutical ingredient having a specific surface area of at least about 5.0 m²/g as measured by B.E.T. comprising:
a) storing the active pharmaceutical ingredient at a temperature of below about 0° C.; and
b) micronizing the active pharmaceutical ingredient to obtain a specific surface area of at least about 5.0 m²/g.

2. The process of claim 1, wherein the storing is carried out at a temperature of about −10° C. to about −20° C.

3. The process of claim 1, wherein the storing is carried out for at least about 4 hours.

4. The process of claim 3, wherein the storing is carried out for at least about 24 hours.

5. The process of claim 1, further comprising the step of storing the micronized active pharmaceutical ingredient at a temperature of below about −10° C.

6. The process of claim 5, wherein the temperature is of about −10° C. and −20° C.

7. The process of claim 5, wherein the storing is carried out for at least about 4 hours.

8. The process of claim 7, wherein the storing is carried out for at least about 24 hours.

9. The process of claim 5, further comprising re-micronizing.

10. The process of claim 9, wherein the re-micronizing yields an active pharmaceutical ingredient having S.S.A. of about 6.0 m²/g to about 7.0 m²/g, as measured by B.E.T.

11. The process of claim 1, wherein mironization is carried out with a feed rate of about 4 kg/hr to about 30 kg/hr.

12. The process of claim 11, wherein the feed rate is about 20 kg/hr.

13. The process of claim 1, wherein mironizing is carried out with a feed air pressure of about 2 bar to about 10 bar.

14. The process of claim 13, wherein the feed air pressure is of about 8 bar to about 8.5 bar.

15. The process of claim 1, further comprising re-micronizing.

16. The process of claim 15, wherein the re-micrinizing yields and active pharmaceutical ingredient having a specific surface area of about 6.0 m²/g to about 7.0 m²/g, as measured by B.E.T.

17. The process of claim 1, wherein the active pharmaceutical ingredient has low bioavailability or is formulated for extended release.

18. The process of claim 17, wherein the active pharmaceutical ingredient is selected from the group consisting of nifedipine and leuprolide.

19. The process of claim 18, wherein the active pharmaceutical ingredient is nifedipine.

20. The process of claim 18, wherein the process results in nifedipine with a specific surface area of at least about 5.5 m²/g.

21. The process of claim 1, wherein the active pharmaceutical ingredient is administered by inhalation.

22. The process of claim 21, wherein the active pharmaceutical ingredient is salmeterol.

23. A process for preparing nifedipine having a specific surface area of at least about 5.0 m²/g as measured by B.E.T. comprising:
a) storing nifedipine powder for a first time at a temperature below about 0° C. for at least about 4 hours;
b) micronizing the nifedipine for a first time to obtain a specific surface area of about 5.0 m²/g to about 6.0 m²/g, as measured by B.E.T.;
c) storing the nifedipine for a second time at a temperature below about −10° C.; and
d) micronizing the nifedipine of step c for a second time to obtain an specific surface area of about 6.0 m²/g to about 7.0 m²/g, as measured by B.E.T.

24. The process of claim 23, wherein the storing is carried out at a temperature of about −10° C. to about −20° C.

25. The process of claim 23, wherein the storing is carried out for at least about 24 hours.

26. The process of claim 23, wherein mironization is carried out with a feed rate of about 4 kg/hr to about 30 kg/hr.

27. The process of claim 26, wherein the feed rate is about 20 kg/hr.

28. The process of claim 23, wherein mironizing is carried out with a feed air pressure of about 2 bar to about 10 bar.

29. The process of claim 28, wherein the feed air pressure is of about 8 bar to about 8.5 bar.

30. A process for maintaining specific surface area of an active pharmaceutical ingredient having a specific surface area of at least about 5.0 m²/g as measured by B.E.T. comprising the step of storing the active pharmaceutical ingredient at a temperature of below about −10° C., wherein the active pharmaceuticcal ingredient retains a specific surface area within about 0.5 m²/g after at least about six months, as measured by B.E.T.

31. The process of claim 30, wherein the active pharmaceutical ingredient is selected from the group consisting of nifedipine, leuprolide and salmeterol.

32. The process of claim 30, wherein the storing is carried out at a temperature of between about −10° C. and about −20° C.

33. The process of claim 30, wherein the storing is carried out for at least about 24 hours.

34. The process of claim 30, wherein mironization is carried out with a feed rate of about 4 kg/hr to about 30 kg/hr.

35. The process of claim 34, wherein the feed rate is about 20 kg/hr.

36. The process of claim 30, wherein mironizing is carried out with a feed air pressure of about 2 bar to about 10 bar.

37. The process of claim 36, wherein the feed air pressure is of about 8 bar to about 8.5 bar.

38. A process for preparing a pharmaceutical oral dosage form comprising:
a) storing an active pharmaceutical ingredient having a particle size distribution of about 15 to about 30 icrons for a first time at a temperature of below about 0° C. for at least about 4 hours;
b) micronizing the stored active pharmaceutical ingredient for a first time to obtain an S.S.A. of at least about 5.5 m²/g, as measured by B.E.T.;
c) storing the active pharmaceutical ingredient for a second time at a temperature of below about −10° C.;
d) micronizing the active pharmaceutical ingredient for a second time to obtain a specific surface area of at least about 6.5 m²/g, as measured by B.E.T.;
e) storing the active pharmaceutical ingredient at a temperature of below about −10° C. for a second time; and
f) converting the active pharmaceutical ingredient to a pharmaceutical oral dosage form.

39. The process of claim 38, wherein the active pharmaceutical ingredient is selected from the group consisting of nifedipine, leuprolide and salmeterol.

40. The process of claim 38, wherein the storing is carried out at a temperature of between about −10° C. and about −20° C.

41. The process of claim 38, wherein the storing is carried out for at least about 24 hours.

42. The process of claim 38, wherein mironization is carried out with a feed rate of about 4 kg/hr to about 30 kg/hr.

43. The process of claim 42, wherein the feed rate is about 20 kg/hr.

44. The process of claim 38, wherein mironizing is carried out with a feed air pressure of about 2 bar to about 10 bar.

45. The process of claim 44, wherein the feed air pressure is of about 8 bar to about 8.5 bar.

46. A process for preparing an active pharmaceutical ingredient selected from the group consisting of nifedipine, salmeterol and leuprolide, having a specific surface area of at least about 5.5 m²/g as measured by B.E.T. comprising:
a) storing the active pharmaceutical ingredient at a temperature of below about 0° C. for at least about 4 hours; and
b) micronizing the stored active pharmaceutical ingredient.

47. The process of claim 46, wherein the active pharmaceutical ingredient is nifedipine.

48. The process of claim 47, wherein micronization is carried out with a feed rate of about 4 kg/hr to about 30 kg/hr and a feed air pressure of about 2 bar to about 10 bar.

49. The process of claim 48, wherein the feed rate is about 20 kg/hr and the feed air pressure is of about 8 bar to about 8.5 bar.

50. The process of claim 48, further comprising storing the micronized nifedipine at a temperature of below about −10° C.

51. The process of claim 50, further comprising converting the obtained nifdipine into formulation.

52. A process for preparing an active pharmaceutical ingredient comprising:
a) storing the active pharmaceutical ingredient at a temperature of below about 0° C. for at least about 24 hours; and
b) micronizing the active pharmaceutical ingredient, wherein the storing results in a minimum increase of about 0.5/g2 in specific surface area compared to micronizing without storing.

53. The process of claim 52, wherein the temperature is of about −10 to about −20° C.

54. A process for preparing a pharmaceutical oral dosage form comprising:
a) storing an active pharmaceutical ingredient for a first time at a temperature of below about negative 10° C. for at least about 24 hours;
b) micronizing the stored active pharmaceutical ingredient at a feed rate of about 20 kg/hr and a feed air pressure of about 8 bar to about 8.5 bar for a first time to obtain an S.S.A. of at least about 5.5 m²/g, as measured by B.E.T.;
c) storing the active pharmaceutical ingredient for a second time at a temperature of below about −10° C.;
d) micronizing the stored active pharmaceutical ingredient for a second time at a feed rate of about 20 kg/hr and a feed air pressure of about 8 bar to about 8.5 bar to obtain an S.S.A. of at least about 6.5 m²/g, as measured by B.E.T.;
e) storing the active pharmaceutical ingredient at a temperature of below about −10° C. for a second time; and
f) converting the active pharmaceutical ingredient to a pharmaceutical oral dosage form.
